(19) United States
(12) Patent Application Publication
(10) Pub. No.: US 2026/0295277 A1
(43) Pub. Date: Oct. 1, 2026

(54) ACOUSTIC MODULATION DEVICE FOR MEDICAL APPLICATIONS AND METHOD THEREOF

(71) Applicant: Jacob Warren OWENS, Cape Girardeau, MO (US)

(72) Inventor: Jacob Warren OWENS, Cape Girardeau, MO (US)

(21) Appl. No.: 19/252,207

(22) Filed: Jun. 27, 2025

Related U.S. Application Data
(60) Provisional application No. 63/781,433, filed on Apr. 1, 2025.

(51) Int. Cl. A61N 1/372; A61N 1/05; A61N 1/362; A61N 1/37

(52) U.S. Cl. CPC A61N 1/37217; A61N 1/0541; A61N 1/3627; A61N 1/3712

(57) ABSTRACT

An acoustic modulation device for thrombus prevention may be provided. The device may have at least one ring removably connected to a catheter with a catheter lumen, a plurality of dual-frequency oscillators that emit acoustic ultrasound waves, a plurality of internal transducers in contact with the catheter that transmit the acoustic waves therein, a microcontroller configured to transmit and receive data wirelessly and allow remote tuning of acoustic frequencies, and a plurality of smart sensors in contact with the catheter and configured to transmit data to the microcontroller.

### BACKGROUND

Central venous catheters (CVCs) are commonly used in medical settings for delivering medications, fluids, and nutrition. However, thrombus formation within the catheter lumen can lead to life-threatening complications, including occlusion, infection, and catheter-related bloodstream infections (CRBSI), also known as central line-associated bloodstream infections (CLABSI). The Centers for Disease Control and Prevention (CDC) estimates that CLABSI contributes to approximately 250,000 infections annually in the United States. CLABSI is associated with a mortality rate ranging from 12% to 25% and the financial burden of each CLABSI case ranges from $25,000 to $45,000, leading to billions in additional healthcare costs.

Thrombus formation occurs when blood is allowed to remain stagnant within the catheter lumen and begins to coagulate. Anticoagulants such as Heparin and saline flushes are commonly used chemical techniques to prevent this. However, chemical approaches may increase bleeding risk in the patient and require repeated administration.

Another technique involves coating the catheter with silver to prevent bacterial buildup, or coating it with Heparin or a drug-eluting coating. Passive approaches like catheter coatings vary in effectiveness and may still increase the risk of bleeding.

More active techniques include low power mechanical vibration devices and external non-targeted ultrasound devices such as Doppler or IV therapy. However, these methods lack precision and dynamic frequency control, which limits their applicability and effectiveness. Ultrasound devices are further limited by external application, which reduces their ability to effectively target thrombus formation.

### SUMMARY

An acoustic modulation device for thrombus prevention may be provided. The device may have at least one ring removably connected to a catheter with a catheter lumen, a plurality of dual-frequency oscillators that emit acoustic ultrasound waves, a plurality of internal transducers in contact with the catheter that transmit the acoustic waves therein, a microcontroller configured to transmit and receive data wirelessly and allow remote tuning of acoustic frequencies, and a plurality of smart sensors in contact with the catheter and configured to transmit data to the microcontroller.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

FIG. 1 shows an exemplary acoustic modulation device.

FIG. 2 shows a flow diagram of an exemplary method performed by an exemplary embodiment of the acoustic modulation device.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

Further, many of the embodiments described herein are described in terms of sequences of actions to be performed by, for example, elements of a computing device. It should be recognized by those skilled in the art that the various sequence of actions described herein can be performed by specific circuits (e.g., application specific integrated circuits (ASICs)) and/or by program instructions executed by at least one processor. Additionally, the sequence of actions described herein can be embodied entirely within any form of computer-readable storage medium such that execution of the sequence of actions enables the processor to perform the functionality described herein. Thus, the various aspects of the present invention may be embodied in a number of different forms, all of which have been contemplated to be within the scope of the claimed subject matter. In addition, for each of the embodiments described herein, the corresponding form of any such embodiments may be described herein as, for example, a computer configured to perform the described action.

Many of the embodiments described herein are described in terms of sequences of actions to be performed by, for example, an Artificial Intelligence (AI) module or modules. It will be understood by those skilled in the art that the sequence of actions described herein can be embodied entirely within any form of AI or ML architecture such that execution of the sequence of actions enables the processor to perform the functionality described herein. Thus, the various aspects of the present invention may be embodied in a number of different forms, all of which have been contemplated to be within the scope of the claimed subject matter. For example, machine learning architectures include but are not limited to Artificial Neural Networks (ANNs), Multi-Layer-Perceptrons (MLPs), Support Vector Machines (SVMs), Recurrent Neural Networks (RNNs), Large Language Models (LLMs), transformers, decision trees, random forests, gradient boosting, expert systems, mixture of experts models, ensemble models, diffusion models, reinforcement learning, and autoencoder models, to name a few. However, many other forms of AI and ML architectures that enable the processor to perform the same functionality have been considered.

It may generally be contemplated for any AI or machine learning architecture to be retrained according to the data processed herein, for example automatically or continuously retrained on a predetermined schedule or based on one or more triggers, such as based on one or more detected changes in the data.

It may be contemplated for execution of the sequence of actions contemplated to be undertaken by the AI or ML architecture to be based on data retrieved from any sensor contemplated herein, and for execution of the sequence of actions to include actuation of any of the one or more transducers contemplated herein.

In one or more exemplary embodiment an acoustic modulation device and method may be provided.

It can be noted that as used herein and in the appended claims, the singular forms “a,” “an,” and “the” include plural references unless the context clearly dictates otherwise. Although any devices and methods similar or equivalent to those described herein can be used in the practice or testing of embodiments, only some exemplary devices and methods are now described.

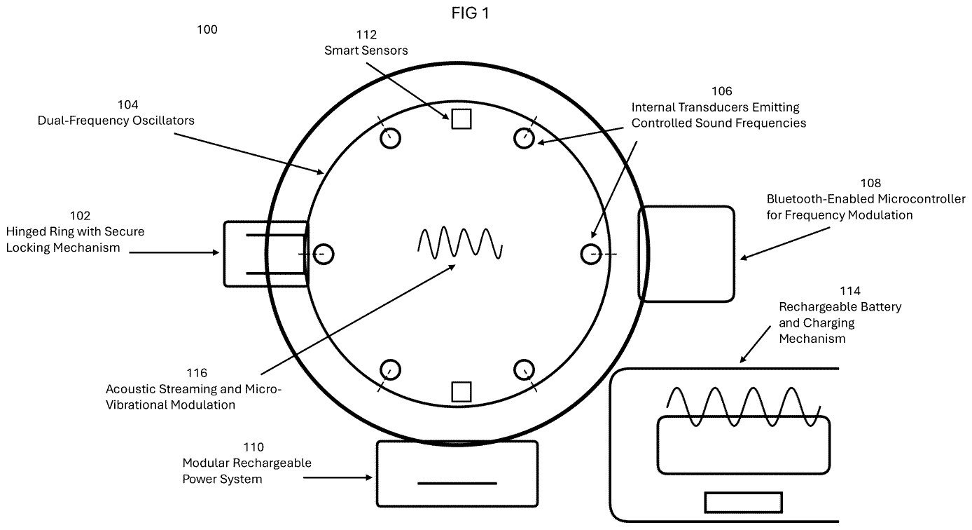

FIG. 1 is an exemplary acoustic modulation device. The acoustic modulation device 100 may include a hinged ring with secure locking mechanism 102. In some embodiments, the hinged ring and secure locking mechanism 102 may include a rectangular structure and/or fasteners. These fasteners may secure the acoustic modulation device 100 to the external portion of a central venous catheter (CVC). In some embodiments, the fasteners may include elastic Velcro or rubber straps, clips, and/or other mechanical, friction based, or adhesive based connectors. In some embodiments, the hinged ring with secure locking mechanism 102 may be adjustable to allow it to be securely fastened around different types of catheters with variable diameters.

In some embodiments, the hinged ring and secure locking mechanism 102 may include an adjustable ring housing constructed out of, for example, PVC pipe, flexible silicone, TPU, 3D-printed casing, and/or malleable plastic. In some exemplary embodiments, the hinged ring and secure locking mechanism 102 may include a plastic enclosure for protecting electronic components and/or padding for protecting the catheter from occlusion.

The acoustic modulation device 100 may further include dual frequency oscillators 104. In an exemplary embodiment, these dual frequency oscillators 104 may be piezoelectric oscillators. In another embodiment, they may be MEMS oscillators. A combination of various types of dual frequency oscillators may also be used. In some embodiments, the dual frequencies of the oscillators may range from, for example, 20 kHz to 3 MHz. In some embodiments, single frequency oscillators may be used. In other embodiments, the dual frequency oscillators 104 may produce a high frequency acoustic wave synchronously with a low frequency acoustic wave. The dual frequency acoustic waves may optimize clot prevention. It may be noted that the dual or single frequencies of some oscillators may, in some embodiments, differ from the frequencies of other oscillators. In some embodiments, a single oscillator may also be used.

The acoustic modulation device 100 may further include internal transducers emitting controlled sound frequencies 106. In some embodiments, the internal transducers 106 may convert electrical signals into targeted acoustic waves which may, for example, prevent thrombus formation. In some embodiments, the internal transducers 106 may amplify, modify, shape, focus, transmit, and/or relay acoustic waves from the dual frequency oscillators 104 or from another source into the catheter lumen or into another target. The internal transducers 106 may, in some embodiments, use piezoelectric or MEMS-based transduction. In an exemplary embodiment, the internal transducers 106 may be evenly spaced around the inner circumference of the inner ring. In some embodiments, the inner ring may be a sound-emitting ring which interfaces with the surface of the catheter lumen. The internal transducers 106 may be, in some embodiments, mounted to the inner ring with a concave or flexible transducer mount to conform to the catheter's cylindrical shape. A wrap-around design that puts the internal transducers 106 in contact with the full circumference of the catheter may also be contemplated.

The acoustic modulation device 100 may further include a wirelessly enabled microcontroller for frequency modulation 108. In an exemplary embodiment, the Bluetooth-enabled microcontroller for frequency modulation 108 may allow for remote tuning of acoustic frequencies via Internet of Things (“IoT”) connectivity. In some embodiments, the microcontroller 108 may also include Wi-Fi capability.

The acoustic modulation device 100 may further include a modular rechargeable power system 110. In an exemplary embodiment, the modular rechargeable power system 110 may provide power to the dual frequency oscillators 104, the internal transducers emitting controlled sound frequencies 106, and/or the Bluetooth-enabled microcontroller for frequency modulation 108. In other embodiments, the modular rechargeable power system 110 may also provide power to other electrical systems or components. In some embodiments, the modular rechargeable power system 110 may, for example, draw power via inductive charging, internal or external battery, and/or USB charging.

The acoustic modulation device 100 may further include smart sensors 112. In an exemplary embodiment, the smart sensors 112 may be located inside the inner ring, near the catheter interface. In some exemplary embodiments, the smart sensors 112 may collect and provide data in the form of temperature, flow rate, flow dynamics, flow characteristics, blood composition, pH balance, and/or bacterial metabolites such as quorum sensing molecules and endotoxins. In some embodiments, other signifiers of, or correlating factors with microbial growth, local infection, inflammation, and/or catheter occlusion/stagnation may also be used as data collected by the smart sensors 112.

The acoustic modulation device 100 may further include a rechargeable battery and charging mechanism 114. In an exemplary embodiment, the rechargeable battery and charging mechanism 114 may provide power to the modular rechargeable power system by means of inductive charging, USB charging, or other cables or wires.

In an exemplary embodiment, the acoustic modulation device 100 may use ultrasonic acoustic frequencies to induce acoustic streaming, resonance effects, micro-vibrations, and/or controlled cavitation within the lumen of a catheter. The acoustic streaming and micro-vibrational modulation 116 may be produced by the dual frequency oscillators 104 and the internal transducers emitting controlled sound frequencies 106. To ensure acoustic transmission efficiency, some embodiments may use a coupling medium between the internal transducers 106 and the catheter surface. This coupling medium may be, for example, medical grade ultrasound gel, sterile saline solution, silicone based acoustic coupling pads, and/or thin medical grade adhesive gel tape. In some embodiments, the coupling medium may be spread as a thin, even layer to prevent the formation of air bubbles.

In some embodiments, the acoustic streaming and micro-vibrational modulation 116 enhances thrombus disruption, prevents thrombus formation, and prevents stagnation of fluid within the lumen of the catheter. The acoustic streaming and micro-vibrational modulation 116 may also serve to reduce the risk of bacterial infection directly and/or indirectly.

In some exemplary embodiments, the acoustic modulation device 100 may also include a computing or Artificial Intelligence (AI) module. In some embodiments these modules may be the same as the Bluetooth-enabled microcontroller for frequency modulation 108, while in other embodiments, one or more of these modules may be separate and distinct.

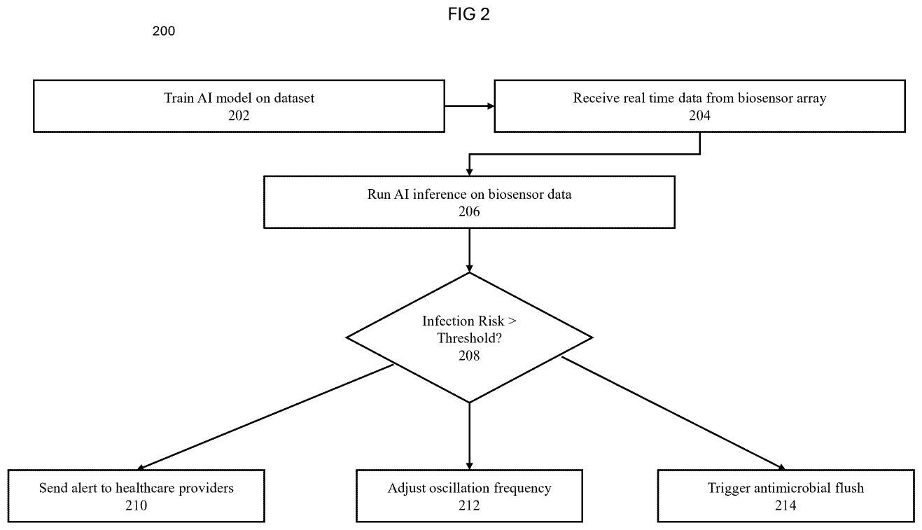

FIG. 2 shows a flow diagram of an exemplary method 200 performed by an exemplary embodiment of the acoustic modulation device. In this example, the embodiment may include an AI or computing module, which includes one or more AI or Machine Learning (ML) models for detecting and quantifying infection risk. In a first step 202 of this exemplary embodiment, the AI or ML model may be trained on a relevant dataset. In a second step 204, the AI or computing module may receive real time data from a biosensor array, such as the smart sensors 112. In a third step 206, the real time data may be passed to the AI or ML model and inference may be run. In this step, the model may produce output data in the form of a prediction value. In a fourth step 208, the output of the AI or ML model may be compared against a predetermined threshold quantity. If the output exceeds this threshold quantity, the AI or computing module may initiate some or all of the following steps. These steps may be performed simultaneously, or in a desired order. In a next step 210, an alert may be sent to healthcare providers. The alert may contain various data, diagnostics, and/or suggested actions. In a next step 212, oscillation frequency may be adjusted. This may serve to disrupt bacterial adhesion and biofilm formation. In a next step 214, an anti-microbial flush may be triggered. In some embodiments, an anti-microbial flush component may be included in the acoustic modulation device 100 and may be used to deliver anti-microbial solutions. In other embodiments, this component may be replaced by or used in conjunction with an electrostatic pulse generator or other antimicrobial countermeasure device.

In some embodiments, the invention may be directed to thrombus prevention and dissolution. In other embodiments, the invention is contemplated as having embodiments such as, for example, a system utilizing acoustic waves to enhance the penetration and distribution of medications at the cellular level, a wearable or non-invasive device that accelerates tissue regeneration using acoustic fluid biophysics, a device designed to prevent deep vein thrombosis (DVT) by optimizing blood flow via acoustic stimulation, a catheter with built-in acoustic modulation to prevent clot formation and improve filtration efficiency, a device targeting neurodegenerative diseases by using specific frequencies to promote neural plasticity and circulation, a non-invasive acoustic therapy designed to disrupt cancerous tumors through resonance and microfluidic stimulation, a method of using acoustic vibrations to enhance lung clearance and improve oxygenation in patients with pulmonary conditions, a sonic toothbrush or oral irrigator that utilizes specific frequencies to break down plaque and bacteria biofilms, and/or a technique using acoustic modulation to enhance the differentiation and proliferation of stem cells for regenerative medicine.

In yet another embodiment, the invention may be directed to treatment of emergency cardiac events such as ventricular fibrillation, asystole, pulseless electrical activity, syncope and peri-arrest states, and/or post-resuscitation neuroprotection. In place of or in conjunction with traditional electrical defibrillation, the invention may be used to induce or restore coherent myocardial contractions, enhance blood flow via acoustic microstreaming, stimulate baroreceptor and vagal pathways and/or modulate the modulate the cardiac-neural field via fluidic resonance. The use of the invention for long term cardiovascular support may also be contemplated. In some embodiments, the invention may emit low power, precisely calibrated acoustic frequencies into a central veinous catheter to modulate hemodynamics, cellular resonance, and systemic coherence. These frequencies may range from 20 Hz to 3 MHz, and in some embodiments, pulsed 605 Hz frequencies may be used to entrain cardiac tissues via mechanoelectrical feedback, restore coherent function of SA/AV nodal networks, interface with the Purkinje system and intrinsic myocardial conduction, modulate parasympathetic tone and promote autonomic rebalancing, and/or preserve or restore consciousness through biofield coherence entrainment.

In a further embodiment, the invention may be used to treat septic or shock states by improving systemic circulation and cellular resonance. In various other embodiments, the invention may also be contemplated as a sedation support tool by stabilizing vagal tone and reducing stress hormones, a stroke intervention tool by enhancing collateral circulation and tissue repair fields, and/or a consciousness recovery tool by reestablishing gamma/theta oscillatory balance through peripheral frequency entrainment.

In some embodiments, the invention may be used at 605 Hz or an equivalent frequency. For example, the invention may be used to entrain cardiac rhythm during asystole, PEA, or post-resuscitation states, modulate stem cell differentiation and biofield regeneration, and/or stabilize quantum superpositions by minimizing field collapse in toroidal coherence nodes. The invention may also be embedded in a diagnostic imaging modality to visualize field symmetry or energetic stagnation and/or applied through wearable or environmental emitters in pulsed geometric sequences for coherence optimization.

In some embodiments, the invention may be used as a propulsion device. For example, using a resonant frequency modulator and an oscillatory transducer array, the invention may create high power standing waves in air, water, or plasma, allowing for aquatic, aerial, or space transportation. Vibrational harmonics may also be controlled to reduce air or fluid resistance and improve efficiency. Acoustic resonance propulsion engines may create pressure gradients to induce thrust without reliance on combustion, chemical reactions, or propellants. In some embodiments, acoustic propulsion engines may also include at least one directional control feedback loop to regulate movement through air, or fluid.

In some embodiments, the invention may include an adaptive acoustic fluid biophysics therapeutic platform. The therapeutic platform may include a plurality of sensors and/or user input to collect biometric, emotional, and environmental data. The therapeutic platform may also include an acoustic fluid biophysics resonance modulation engine, which may deliver acoustic, magnetic, optical, or vibrational frequencies to a patient. The therapeutic platform may include an AI module with at least one artificial intelligence or machine learning model therein. The AI module may be trained and configured to predict patient outcomes and/or to regulate treatment. For example, the AI module may receive real time data from the sensors and output a frequency and/or duration recommendation. This recommendation may then be used to deliver an acoustic or other frequency to the patient via wearable rings, pads, beds, ambient field generators, software based visual-audio systems, and/or other delivery mechanisms. In some embodiments this may include a continual feedback loop that persists until a physician or the AI module determines a stopping point, or until a predetermined stopping point. In some embodiments, the therapeutic platform may also detect stagnation zones and/or modulate geometric structures such as, for example, toroidal or quaternary geometric structures.

In some embodiments, emission or pulsing of audio, ultrasonic, infrasonic, or subharmonic frequencies, including those centered near or around 605 Hz or any other coherence-associated range may facilitate systemic entrainment. Functional frequencies may be delivered by means of geometric field structures. Geometric field structures may include structures such as, for example, Quaternary Coherence Field (4-petal), which may align with 605 Hz and support heart-centered electromagnetic and consciousness coherence, Hexagonal Entrainment Matrix (6-petal, Flower of Life seed, Vesica Piscis Field, Golden Ratio Spiral, Tetrahedral Field, Octahedral Field, Dodecahedral Resonance Grid, Toroidal Field Structure, Metatron's Cube Geometry, Yin-Yang Matrix, Triune Spiral Field, Sacred Octagon (8-Fold Lotus Field), or other known geometric field structures. Toroidal field alignment may facilitate phase-locking between cardiac, neural, and fluidic oscillations. It may also serve as a transition frequency for initiating dimensional field for initiating dimensional field shifts or intentional state modulation (i.e., quantum jumping).

Selected geometric field structures may be physically etched, virtually projected, or dynamically generated in response to biometric or environmental feedback, and may be used to modulate and deliver resonance into a biological, energetic, or quantum computational system. In some embodiments, multiple geometric fields may be layered or sequenced in a dynamic protocol for harmonic optimization.

In some embodiments, the invention may be used for water purification, atmospheric water harvesting, microbial sanitation, or to stimulate soil vitality, seed development, or plant coherence. It may be understood that structured waveform output between 20 Hz and 3 MHz or other equivalent frequencies may stimulate chloroplast activity, increase ATP synthesis, promote growth factor signaling, and affect seed germination, root density, cellular respiration, water structuring, and/or nutrient uptake. Frequencies such as, for example, 605 Hz may influence hydrogen bonding and fluid structuring around roots, enhancing hydration and trace mineral bioavailability. Waveform exposure may harmonize soil microbial communities, increasing nitrogen-fixing bacteria and reducing harmful pathogens through coherent vibrational fields. In some embodiments, modulated acoustic fields may disrupt insect mating cycles, sensory orientation, and nesting behaviors without harming beneficial species.

In some embodiments, an acoustic fluid biophysics agricultural system may be deployed as a stationary field unit mounted on poles for open farmland, a greenhouse ceiling-mounted grid of emitters, a hydroponic root-zone system with direct coupling, and/or a mobile drone-mounted emitter array for fieldwide propagation. Units may include programmable waveform libraries for plant species, growth phase, soil type, and pest class. Power sources may include solar, battery, or grid-tied modules. In some embodiments, the agricultural system may be controlled or informed by an AI module.

In some embodiments, the invention may include an acoustic fluid biophysics based water sanitation system. The system may produce ultrasound-induced cavitation effects that generate microbubbles and shockwaves in order to physically rupture microbial membranes and disaggregate biofilms. Toroidal flow structures induced by pulsed frequencies may enhance mixing, oxygenation, and thermal regulation, increasing sanitation efficiency. Exposure to structured frequencies and geometries may influence hydrogen bonding, improving water's bioavailability and energetic properties. Sonic condensation fields and geometrically guided airflow may also enable efficient extraction of airborne moisture with minimal energy input. Spiral, dodecahedral, or lotus-based structural forms may enhance field stability, resonance propagation, and fluid coherence during sanitation or water harvesting.

The water sanitation system may include a vortex chamber, spiral tubing, or toroidal fluidic shell embedded with transducers that emit modulated sound waves. Water or ambient air may be passed through the chamber where acoustic streaming and cavitation are induced. For sanitation, frequencies including 605 Hz and ultrasound may be used to destroy pathogens and break down dissolved toxins. For water harvesting, sonic condensation fields may induce droplet formation and allow collection within a capture basin. Portable units may use battery or solar power and be designed for field, clinical, or disaster-relief settings. A vortex-generating structure may be configured to induce toroidal flow and resonance symmetry. An AI module may control or inform the water sanitation system based on a plurality of sensor data including, for example, water quality, temperature, flow rate, and microbial load data.

In some embodiments, the invention may include a diagnostic imaging system. The imaging system may map coherence fields to enable early detection of imbalances, stress overload, or latent dysfunction before they manifest physically. The system may complement structural imaging with functional and energetic data. Reflected or altered waveform data may reveal density, coherence, and impedance properties of tissues and fluids, enabling diagnostic inference without invasive sensors. Positive health outcomes may correlate with symmetry and flow in energetic and fluidic systems, which may result in disruptions in toroidal field geometry or resonance patterns indicating energetic trauma or pre-pathological states. In some embodiments, sacred geometries may be used as overlays or analysis matrices to detect harmonic deviation, symmetry breakage, or entrainment loss in the biological field.

The imaging system may include a hand-held or wearable emitter-detector array, with embedded acoustic transducers and sensors. A frequency generator may emit structured sound pulses, and the returning signal may be captured and analyzed for resonance distortion or delay. An AI module may process the signal to create a topographical or three-dimensional map of field behavior. Coherence visualization may be displayed in real-time on a connected device, with sacred geometry overlays for enhanced interpretation. The system may be used in wellness centers, clinics, home settings, or integrated into wearable coherence monitors, for example.

In some embodiments, the invention may include a biofield modulation system. The biofield modulation system may include one or more acoustic field emitters configured to emit modulated sound frequencies between 20 Hz and 3 MHz, and a housing structure or wearable form factor incorporating geometric field structures which may include, for example, toroidal, spiral, lotus, or sacred geometry patterns. The acoustic field may include a pulsed 605 Hz frequency configured to modulate consciousness, cardiac coherence, and bioenergetic field symmetry in a human subject. In some embodiments, the biofield modulation system may include a modular ring-shaped device designed to clasp around the external portion of a central venous catheter or wearable contact point. Geometric field modulation may be achieved via embedded patterns within the transducer mount or through pulsed frequency harmonics that reproduce toroidal field symmetry. Frequencies including 605 Hz may be delivered in pulses or standing wave patterns using, for example, Bluetooth-enabled microcontrollers.

Sensors may measure HRV, skin conductivity, or local electromagnetic field to dynamically adjust the acoustic signature. The entire system may be powered by an internal rechargeable battery or by energy-harvesting components. Variants of the device may be configured for meditation headsets, sleep bands, catheter-mounted therapeutic rings, or personal wearable discs.

Specific sound frequencies may be used to affect brainwave states, particularly in the theta, alpha, and gamma bands. Frequencies such as, for example, 528 Hz and 432 Hz may be used to induce relaxation and coherence in a patient. The frequency 605 Hz is positioned between the Solfeggio scale and golden ratio resonances, and may be used to influence higher-order consciousness patterns and coherent physiological entrainment. Heart rate variability (HRV) and vagal nerve stimulation may influence mental state, emotional regulation, and trauma resilience. Acoustic vagal stimulation via auricular or thoracic methods may also be used. The biofield modulation system may use field geometry and acoustic harmonics to enhance coherence between the cardiac and neural systems.

Geometric field structures may be used to influence symmetry, resonance, and stability of energy transmission. Patterns such as, for example, the torus, spiral, and sacred geometries may be used to modulate field propagation in electromagnetic and acoustic domains. These geometries may support stable resonance fields capable of entraining biological systems. By stabilizing peripheral field symmetry (toroidal coherence), the biofield modulation system may facilitate entrainment of subtle consciousness states, reduce fragmentation, and promote neural integration. This may provide a foundation for non-pharmacological consciousness support tools for meditation, sleep, recovery, and trauma repair. An AI module may also be included to personalize frequency and/or field geometry in real time.

In some embodiments, the invention may include a non-electrical emergency cardiac intervention system. The cardiac intervention system may restore cardiac function in emergency situations such as asystole, pulseless electrical activity (PEA), and ventricular fibrillation through mechanoelectric entrainment, toroidal resonance, and vagus-integrated field modulation. The system may utilize an externally applied or catheter-adjacent acoustic emitter configured to deliver pulsed sound frequencies including 605 Hz and other harmonics known to promote myocardial synchronization, baroreceptor stimulation, and coherence re-establishment. The system may offer a non-electrical, non-pharmacological method for reinitiating cardiac rhythm and preserving consciousness during cardiac arrest or peri-arrest states.

Many patients in asystole or PEA do not respond to electrical defibrillation, and even when return of spontaneous circulation (ROSC) is achieved, long-term neurological damage is common due to delayed coherent perfusion and lack of neurocardiac field restoration. However, acoustic waves and low-frequency resonance can influence baroreceptor pathways, vagal tone, cardiac pacemaking, and even mechanoelectrical transduction. Cardiac cells may exhibit mechanoelectrical coupling, where physical vibration or pressure waves influence ionic channels and action potential propagation. Targeted acoustic pulses can entrain these pathways and potentially re-establish pacemaking function in the sinoatrial and atrioventricular nodes.

The vagus nerve, central to autonomic tone and heart rate regulation, can be stimulated through low-frequency acoustic pulses. This supports coherence restoration and neurocardiac synchronization in pre-arrest and post-arrest states. Frequencies such as 605 Hz and other harmonic patterns may be used to promote coherence between heart tissue and central regulatory systems. Pulsed toroidal fields may stabilize oscillatory collapse during fibrillation or arrest. Acoustic cavitation, streaming, and field interference can generate force vectors and energy distributions capable of supporting micro-circulatory entrainment and fluidic propagation of contractile potential. Consciousness loss during arrest may correlate with breakdown of large-scale biofield geometry. AFB systems may preserve or re-establish field integrity, supporting faster neurological recovery.

The cardiac intervention system may include an adjustable acoustic emitter ring or patch with embedded piezoelectric or MEMS-based transducers. The emitter may be configured to clasp around an external catheter segment or be applied to the chest, neck, or sternal notch. The device may emit pulsed acoustic frequencies, for example, 605 Hz and harmonic sequences, via a Bluetooth-enabled controller. An AI module may adjust pulse train patterns based on biosensors that measure ECG activity, thoracic impedance, or baroreflex response. Power may be delivered by internal battery, magnetic induction, or emergency capacitor charge. In some embodiments, the system may interface with defibrillator logic to trigger simultaneous acoustic and electrical protocols. Wearable versions may serve as preventative or early-intervention cardiac support tools.

In some embodiments, the invention may include a modular electrokinetic enhancement system. The electrokinetic enhancement system may include at least two unconnected, opposed-polarity battery nodes mounted on an external acoustic resonance device. The positive terminals of each battery may be oriented toward one another to generate a static electrofield across a fluidic or dielectric zone. This field may interact with ionic media or biological fluids to enhance fluid motion, promote structured flow, and synergize with acoustic streaming and magnetic resonance components. The system may be designed as an optional, non-powered, field-modulating accessory within wearable or catheter-adjacent therapeutic devices.

Electric fields may influence ion transport, induce electrophoretic motion, and stimulate electrohydrodynamic (EHD) flow. However, conventional implementations typically rely on powered electrode systems. The electrokinetic enhancement system may use unconnected battery elements to generate passive field interactions through spatial polarity configuration for therapeutic or biofluidic modulation. In some embodiments, it may include two or more batteries are mounted into a ring-shaped acoustic device such that their positive terminals face one another. These batteries may not be electrically connected to the device circuitry or to each other. The proximity of their terminals may establishe a localized electrostatic gradient between them.

When the electrokinetic enhancement system is placed around a fluid-carrying structure (e.g. a catheter), the electrofield may interact with ionic or polar fluids, inducing, for example, mild ion drift or redistribution, potential electroosmotic or EHD motion, enhanced microstreaming when paired with acoustic and magnetic fields, and/or field symmetry that reinforces toroidal or spiral flow geometry. It may also create local dielectric polarization and/or influence the orientation of water dipoles. In some embodiments, the configuration of the electrokinetic enhancement system may not require current flow and may serve solely to alter field behavior through charge proximity and polarity repulsion.

EHD flow may arise from the interaction between electric fields and mobile charge carriers in fluids. Even weak static fields can drive ion migration, induce charge separation at boundaries, or cause bulk fluid motion via body force distribution per the Coulomb force: F=qE. This may be especially true in small channels or ring-shaped geometries where field gradients are confined. When combined with acoustic transducers producing pressure and streaming waves, as well as magnets generating static or rotating magnetic fields, the electrostatic field from the opposed batteries may reinforce toroidal streaming, prevent stagnation zones, and modulate coherence patterns within or around the catheter lumen. From a field geometry perspective, the placement of symmetrically opposed positive battery terminals may add a stabilizing energetic axis to the device. This may increase balance in acoustic wave propagation, support harmonized oscillation modes, and/or reduce drift or chaotic flow behaviors.

In some exemplary embodiments, the electrokinetic enhancement system may include two standard coin-cell or circular rechargeable batteries that are inserted into diametrically opposite slots in the ring housing. The positive terminals may face inward, aligned along a common radial axis perpendicular to the catheter. These batteries may be modular and may be inserted or removed depending on treatment without a need for wires, leads, or circuits to be connected to them.

In some embodiments, the invention may include an opposed magnetic node system for enhancing the field coherence, resonance geometry, and therapeutic effectiveness of acoustic modulation devices. The opposed magnetic node system may comprise two or more permanent magnets placed in diametrically opposed positions around a ring-shaped acoustic device. The static magnetic fields generated by the magnets may interact with the acoustic and electrokinetic fields to enhance microstreaming, toroidal symmetry, and biofield coherence. In some embodiments, the magnets are not used for electrical input but serve as passive field modulators within a multi-physics therapeutic system. Combining multiple physical fields such as acoustic, electric, and magnetic may be used to amplify biological effects, increase coherence, and support advanced functionalities such as flow control and thrombus disruption.

Static magnetic fields may influence the orientation and behavior of moving charges and polar molecules. In conductive or ionic media, a magnetic field orthogonal to a pressure wave or electrical gradient can induce Lorentz forces, alter ionic paths, and/or create gyroscopic fluid motion. Magnetic fields can be used to modulate the directionality and coherence of acoustic wave propagation. Such combinations may improve resonance focusing, alter absorption patterns in tissues, and/or enhance alignment of field-sensitive materials. Placing opposing magnets on a circular housing may introduce rotational symmetry to the field profile which may stabilize toroidal flow within the catheter, prevent chaotic wave reflection or standing wave collapse, and/or reinforce energetic coherence within fluidic systems. By using passive permanent magnets, the opposed magnetic node system may introduce no additional power burden but may significantly influence the internal field architecture of the therapeutic device. Magnetic contributions may increase the effectiveness of acoustic and electrokinetic systems in promoting flow, coherence, and physiological modulation.

In one exemplary embodiment of the opposed magnetic node system, two neodymium or ferrite permanent magnets may be embedded in modular slots on opposite sides of a ring housing. The magnets may face inward toward the catheter interface. Their field orientation may be parallel or orthogonal to the fluid conduit, depending on intended use. Magnets may be shielded or encased in biocompatible plastic or gel inserts. The system may be designed to be non-powered, passive, and modularly upgradeable.

In some embodiments, the invention may include a non-cryogenic system for qubit coherence stabilization. The stabilization system may use acoustic fluid biophysics (AFB) to stabilize qubit coherence, eliminate decoherence-inducing noise, and facilitate scalable, room-temperature quantum architectures. This system may use structured acoustic frequencies (including, for example, 605 Hz and harmonics), toroidal field symmetry, and/or resonant geometry to shield and regulate quantum states in physical, fluidic, and hybrid quantum platforms.

Quantum computers suffer from instability due to decoherence, the rapid collapse of quantum states under thermal, vibrational, and electromagnetic noise. Solutions such as dilution refrigerators and high-frequency error correction codes introduce complexity, cost, and physical constraints. These approaches often lack scalability, biological compatibility, or real-time coherence adaptation. The stabilization system may use acoustic frequencies from, for example, 20 Hz to 3 MHz to suppress decoherence pathways. It may also include toroidal field structures and geometry-based shielding architectures. Applications to fluidic, topological, superconducting, photonic, and hybrid quantum systems may also be contemplated. In some embodiments, the stabilization system may enable room-temperature coherence extension and passive environmental regulation. In some exemplary embodiments, the stabilization system may use 605 Hz as a fundamental coherence entrainment carrier.

Targeted sound frequencies can suppress environmental phonon interactions, effectively stabilizing quantum superpositions and preventing thermally induced collapse. Geometrically symmetric resonance structures can protect qubits from vibrational and EM flux using nodes of minimal energy variation and harmonic shielding. Additionally, qubits supported in fluidic or hybrid materials may be passively stabilized by frequency-induced flow coherence and vortex symmetry. In some embodiments, AFB shielding may be used to reduce decoherence, extend qubit lifetime and reduce gate errors, thereby minimizing the need for redundant correction protocols and cryogenic environments. In some embodiments, qubit lifetime may be extended beyond conventional T2 decay times.

In some exemplary embodiments, the stabilization system may include an array of piezoelectric emitters arranged around a toroidal housing that surrounds or integrates with a qubit substrate. The emitters may be driven by a programmable microcontroller tuned to specific harmonic frequency sequences. Applications may include, for example, room-temperature topological qubits, superconducting chip encasements, photonic delay-loop stabilizers, or biologically integrated coherence chips. A 3D-printed modular shell containing sacred geometry channels may amplify shielding effects and preserve coherence across computational cycles. In some embodiments, field strength and frequency may be modulated via an AI module based on decoherence signal feedback.

In some embodiments, the invention may include a drug delivery enhancement system. The drug delivery enhancement system may allow for targeted bioavailability using acoustic resonance, fluidic microstreaming, and waveform-based diffusion optimization. The invention may use sound frequencies such as, for example, 20 Hz-3 MHz, and field-symmetric geometries to enhance the penetration, cellular uptake, and tissue absorption of pharmaceutical, nutraceutical, or biologic compounds. Applications may include catheter-assisted infusion, transdermal delivery, oral absorption support, and sonic activation of liposomal or nanoparticle-based therapeutics.

Many current drug delivery techniques are limited by tissue permeability, cellular uptake, and the requirement for invasive delivery routes or chemical carriers. While nanoparticle and liposome technologies have improved targeting, absorption remains inconsistent and highly dependent on the delivery environment. The drug delivery enhancement system may use acoustic field modulation to increase the bioavailability and delivery precision of therapeutic agents. In some embodiments, it may use frequencies from, for example, 20 Hz to 3 MHz for targeted streaming and permeability enhancement. Integration with catheter, patch, oral capsule, or localized emitter forms may also be contemplated. In some embodiments, the system may include support for nanoparticle, liposomal, or biologic payloads, and/or toroidal microstreaming and waveform-driven enhancement of diffusion, convection, and tissue absorption. In some embodiments, the system may include real-time sensing and/or coordination by an AI module for delivery timing and modulation optimization.

Low-to-mid-frequency sound waves may induce fluid movement known as acoustic streaming. This may create local convection currents that enhance molecular diffusion and tissue penetration. Acoustic pressure waves may increase membrane permeability temporarily without causing damage, allowing greater intracellular delivery. Sound waves can be tuned to destabilize nanoparticle carriers at target zones, improving precision unloading of the compound. Structured acoustic fields based on sacred geometry may be used to improve symmetry of delivery and reduce loss via chaotic turbulence. Specific compounds may respond better to different modulation patterns, which may allow for frequency-personalized pharmacoacoustic protocols.

In some exemplary embodiments, the drug delivery enhancement system may include a wearable, catheter-clasp, or patch-mounted system with piezoelectric transducers that emit structured frequencies around the compound delivery site. For intravenous use, the system may clasp externally to the catheter or infusion port, generating microstreaming inside the lumen. The emitter may modulate tone based on preset delivery sequences or adjust in real-time based on biosensors measuring fluid dynamics or tissue impedance. Other exemplary embodiments include, for example, oral capsule activation systems, subcutaneous patch devices, and aerosol or nasal delivery amplifiers.

In some embodiments, the invention may include a regenerative healing system. The regenerative healing system may include activation and differentiation of stem cells, accelerated tissue repair, and/or modulation of morphogenetic fields via geometrically structured acoustic waveforms. The regenerative healing system may also include a therapeutic device using sound frequencies of, for example, 20 Hz-3 MHz, sacred geometric field designs, and/or dynamic resonance patterns to induce localized or systemic biological regeneration. Applications may include wound healing, organ regeneration, neurorepair, orthopedic integration, and vascular restoration.

Regenerative medicine depends on the precise activation and differentiation of stem cells and the restoration of coherent tissue architecture. Current approaches typically involve biochemical cues (e.g., growth factors), scaffold engineering, and stem cell injections. However, cells may also respond to mechanical and acoustic stimuli via mechanotransduction, cytoskeletal activation, and/or epigenetic regulation. The regenerative healing system may use frequencies ranging from, for example, 20 Hz to 3 MHz to stimulate cellular coherence, cytoskeletal remodeling, and/or DNA expression pathways. Field geometries such as toroidal spirals, lotus symmetry, and golden ratio spirals embedded in the emitter structure or pulse sequence may also be used. In some exemplary embodiments, resonance may be applied to localized injuries (wounds, fractures, burns) or to systemic regions (organ repair, brain injury, vascular regeneration). The regenerative healing system may include wearable or catheter-clasp form factors with embedded frequency control, pulse logic, and optional biofeedback sensor systems. In some embodiments, the system may be used with or without exogenous stem cells to promote innate healing, improve graft integration, and accelerate regenerative repair.

Acoustic waves may produce micro-vibrations and streaming patterns that influence cell membrane ion channels, cytoskeletal filaments, and epigenetic transcription regulators. This may support lineage specification and proliferative activity in pluripotent and mesenchymal stem cells. Sacred geometries (e.g., torus, Metatron's Cube, golden spiral) may generate stable symmetry fields that facilitate may morphogenesis. Applying these geometries via structured sound may be used to enhance signal fidelity and tissue organization. Specific frequencies may influence gene expression through vibratory energy fields. Pulsed acoustic fields may promote differentiation toward desired lineages (e.g., osteogenic, neurogenic, myogenic). AFB may be used to modulate inflammation, fluid stagnation, and ischemia at injury sites, improving local regenerative potential and reducing scarring or fibrosis. Unlike biochemical or scaffold-based methods, some embodiments of the regenerative healing system may use resonance to organize cellular behavior directly via physics-based principles, making it ideal for non-invasive, portable, or field-based therapy.

In some exemplary embodiments, the regenerative healing system may include a wearable acoustic emitter with embedded piezoelectric transducers arranged in a quaternary or toroidal field pattern. The emitter may be powered by a microcontroller programmed with healing-specific frequency profiles, including but not limited to 528 Hz, 605 Hz, and higher harmonics. In some embodiments, the regenerative healing system may be placed over wounds, joints, surgical sites, or neural structures. A silicone or gel coupling interface may be used to enhance wave transmission. Biosensors may provide feedback on tissue impedance, temperature, or inflammation to dynamically modulate therapy. Optional integration with stem cell delivery systems may allow synchronized regenerative activation at the cellular and field levels.

Although specific structures and components are described, the invention is not limited to the exact arrangements shown. Equivalent systems—external, internal, or adjacent—that perform substantially the same function in substantially the same way to achieve the same result are within the scope. Variants may include open-loop operation, manual frequency control, or preset modulation instead of AI or sensor input. Alternate geometries, wearable forms, or fluidic systems operating by acoustic modulation for thrombus prevention, infection control, or cardiac support are likewise included.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. An acoustic modulation device for thrombus prevention comprising:
at least one ring, being removably connected to a catheter, the catheter having a catheter lumen;
a plurality of dual-frequency oscillators configured to emit acoustic waves in an ultrasound range;
a plurality of internal transducers being in contact with a surface of the catheter and being configured to transmit the acoustic waves therein;
a microcontroller, the microcontroller being configured to transmit and receive data wirelessly; wherein the microcontroller is configured to allow remote tuning of acoustic frequencies; and
a plurality of smart sensors being in contact with a surface of the catheter and being configured to transmit data to the microcontroller.

2. The acoustic modulation device of claim 1, wherein the dual-frequency oscillators are configured to emit the acoustic waves in both a low frequency and a high frequency, the low frequency ranging from 20 kHz-100 kHz, and the high frequency ranging from 100 kHz-3 MHz.

3. The acoustic modulation device of claim 2, wherein the dual-frequency oscillators are configured to emit the low frequency acoustic waves and the high frequency acoustic waves synchronously.

4. The acoustic modulation device of claim 1, wherein the internal transducers are piezoelectric or MEMS-based transducers.

5. The acoustic modulation device of claim 1, further comprising a rechargeable battery.

6. The acoustic modulation device of claim 1, wherein the catheter is a central venous catheter.

7. The acoustic modulation device of claim 1, wherein the ring comprises a secure locking mechanism.

8. The acoustic modulation device of claim 1, further comprising an AI module, the AI module comprising a plurality of machine learning algorithms.

9. The acoustic modulation device of claim 1, further comprising an anti-microbial flush component configured to deliver an anti-microbial solution into the catheter lumen.

10. A method of acoustic modulation, comprising the steps of
training a plurality of machine learning algorithms on a dataset;
collecting real time data from a patient with a plurality of sensors attached to a catheter, the catheter having a catheter lumen and a plurality of dual-frequency oscillators configured to emit acoustic waves in an ultrasound range;
inputting the real time data into the plurality of machine learning algorithms and receiving as output an infection risk prediction value;
comparing the infection risk prediction value against a predetermined threshold value;
transmitting a signal to a microcontroller if the infection risk prediction value exceeds the predetermined threshold value;
modulating the frequency of the plurality of dual-frequency oscillators based on the signal.

11. The method of claim 10, further comprising
sending an alert to at least one healthcare provider based on the signal.

12. The method of claim 10, further comprising
triggering an anti-microbial flush within the catheter lumen.
